(19) United States
(12) Patent Application Publication
(10) Pub. No.: US 2004/0265961 A1
(43) Pub. Date: Dec. 30, 2004

(54) Novel proteins homologous to kinase suppressor of Ras

(76) Inventors: Wei Liu, Sudbury, MA (US); Leeying Wu, Lexington, MA (US); Padma L. Channavajhala, Woburn, MA (US); Lih-Ling Lin, Concord, MA (US); Yuhua Zhang, Wellesley, MA (US)

Correspondence Address: FITZPATRICK CELLA (WYETH), 30 ROCKEFELLER PLAZA, NEW YORK, NY, 10112-3800, US

(21) Appl. No.: 10/812,232

(22) Filed: Mar. 29, 2004

Related U.S. Application Data
(60) Provisional application No. 60/457,928, filed on Mar. 28, 2003.
(60) Provisional application No. 60/491,283, filed on Jul. 31, 2003.

(51) Int. Cl. C12N 9/12; C07H 21/04

(52) U.S. Cl. 435/69.1; 435/194; 435/320.1; 435/325; 536/23.2

(57) ABSTRACT

The invention provides isolated protein kinase polypeptides homologous to Kinase Suppressor of Ras (KSR), and the isolated nucleic acid molecules that encode these polypeptides. The novel polypeptides and nucleic acid molecules of the invention are termed KSR-2. The invention also provides genetically engineered expression vectors, host cells, and transgenic animals comprising the novel nucleic acid molecules of the invention. The invention additionally provides antisense and RNAi molecules to the nucleic acid molecules of the invention. The invention further provides inhibitors, activators, and antibodies capable of binding to the protein kinase polypeptides of the invention. The invention also provides for the regulation of Cot/Tp12-mediated cellular functions, such as ERK and NF-κB activation, as well as IL-8 production, through the use of the nucleic acid and protein kinase polypeptide molecules of the invention.

This application claims the benefit of U.S. Provisional Application Ser. No. 60/457,928, filed Mar. 28, 2003, and U.S. Provisional Application Ser. No. 60/491,283, filed Jul. 31, 2003, both of which are incorporated herein by reference in their entireties.

### BACKGROUND OF THE INVENTION

1. Field of the Invention

The present invention is directed to novel proteins homologous to kinase suppressor of Ras (KSR), as well as nucleic acid molecules encoding such novel proteins. More specifically, the present invention provides novel peptides and proteins that function in the Ras pathway, as well as nucleic acid molecules encoding such peptides and proteins.

2. Related Background Art

The Ras pathway plays a critical role in the transmission of many growth and developmental signals. A major route by which Ras transmits these signals is through the sequential activation of various cytoplasmic kinases, including Raf, MEK and MAP kinase (MAPK).

Recently, large-scale genetic screens performed in Drosophila and C. elegans led to the isolation of a new component of the Ras pathway, which was termed kinase suppressor of Ras (KSR) (Kornfeld et al. (1995) Cell 83:903-13; Sundaram and Han (1995) Cell 83:889-901; Therrien et al. (1995) Cell 83:879-88). KSR constitutes a protein kinase family that is structurally related to the Raf family of kinases (Sundaram and Han (1995), supra).

The presence of a putative kinase domain in KSR suggested that KSR might act as a kinase in the Ras pathway. This notion was bolstered by the fact that many of the KSR-inactivating mutations identified in Drosophila and C. elegans occur in the kinase domain. In addition, some evidence suggested that KSR is a ceramide- and EGF-activated protein kinase that can phosphorylate Raf-1 on threonine 269 in vitro and can thereby increase the kinase activity of Raf (Zhang et al. (1997) Cell 89:63-72; Xing and Kolesnick (2001) J. Biol. Chem. 276:9733-41).

Several observations, however, have cast doubt on whether KSR functions as a kinase. First, all mammalian KSR proteins contain an arginine residue at a position in the ATP-binding domain that is normally occupied by a lysine residue that is involved in the phosphotransfer reaction and is usually required for enzymatic activity (Therrien et al., supra). Second, unlike in vitro studies, the phosphorylation of Raf on threonine 269 has not been reported in vivo (Morrison (2001) J. Cell Sci. 114:1609-12). Third, KSR constructs that lack the putative kinase domain augment Raf-1 in a kinase-independent manner (Michaud et al. (1999) Proc. Natl. Acad. Sci. USA 94:12792-96). Fourth, KSR proteins containing mutations expected to eliminate kinase activity rescue the KSR loss-of-function phenotype in C. elegans (Stewart et al. (1999) Mol. Cell Biol. 19:5523-34). Finally, the isolated catalytic domain of KSR, rather than enhancing Ras-dependent signaling, acts as a dominant inhibitory protein that blocks Ras signaling and MAPK activation in mammalian, Xenopus, and Drosophila cells (Therrien et al. (1996) Genes Dev. 10:2684-95; Yu et al. (1997) Curr. Biol. 8:56-64; Joneson et al. (1998) J. Biol. Chem. 273:7743-48).

Recent evidence suggests that KSR facilitates the signaling of Ras by acting as a scaffolding protein that coordinates the assembly of a multiprotein complex containing MAPK and its upstream regulators. KSR has been shown to interact with Raf-1, MEK, and MAPK (Roy et al. (2002) Genes Dev. 16:427-38; Muller et al. (2000) Mol. Cell. Biol. 20:5529-39; Xing et al. (1997) Curr. Biol. 7:294-300; Denouel-Galy et al. (1997) Curr. Biol. 8:46-55; Yu et al. (1997), supra) and to translocate from the cytosol to the plasma membrane in response to Ras activation and growth factor treatment (Muller et al. (2001) Mol. Cell. 8:983-93; Michaud et al. (1997), supra). In addition, KSR-deficient mice have attenuated MAPK activation to a degree sufficient to block T-cell activation and tumor development (Nguyen et al. (2002) Mol. Cell. Biol. 22:3035-45). These observations suggest a model in which KSR provides a scaffold that facilitates the phosphorylation reactions that are required for executing critical signal transduction steps downstream of Ras.

Further support for the KSR scaffolding model has come from findings that other accessory proteins involved in the Ras pathway also interact with KSR. Hsp90, Hsp70, Hsp68, p50^{cdc37}, G protein γ subunits and 14-3-3 proteins were shown to interact with KSR and appear to be required for protein stability, as disruption of these interactions results in rapid KSR degradation (Stewart et al. (1999), supra). In particular, 14-3-3 proteins appear to play a critical role in KSR function by binding to two phosphoserine residues, thereby maintaining KSR in an inactive state until activated Ras-induced dephosphorylation occurs (Muller et al. (2001), supra; Cacace et al. (1999) Mol. Cell. Biol. 19:229-40).

The KSR scaffolding model offers an attractive explanation for the observation that the biological effects of KSR vary with the level of KSR protein expressed. When KSR is expressed at low or near physiological levels, it functions as a positive effector of Ras signaling (Therrien et al. (1996), supra; Cacace et al. (1999), supra; Muller et al. (2000), supra). When KSR is highly overexpressed, however, it inhibits Ras signaling in mammalian, Xenopus, and Drosophila cells (Denouel-Galy et al. (1998), supra; Sugimoto et al. (1997) EMBO J. 17:1717-27; Yu et al. (1997), supra; Joneson et al. (1998), supra; Cacace et al. (1999), supra), suggesting that when the level of KSR drastically exceeds the levels of its interacting components, nonfunctional complexes that lack critical components form and inhibit signal transmission (Morrison (2001), supra). The scaffolding model also accounts for the dominant inhibitory effect of the isolated KSR catalytic domain, which appears to sequester MEK in the cytosol, resulting in the short-circuiting of the signal transduction from Ras to MAPK (Muller et al. (2000), supra).

Because the Ras pathway has been shown to play a critical role in both normal and abnormal growth processes, it would be desirable to identify and characterize novel components of this pathway. In particular, because KSR has been shown to play a positive role in Ras signaling by coordinating the formation of a multiprotein kinase complex, it would be desirable to identify and characterize novel members of the KSR family.

Cot (Tp12 in rat), a human protooncogene (Sasai et al. (1993) Br. J. Cancer 67:262-67; Miyoshi et al. (1991) Mol. Cell. Biol. 11:4088-96), is a serine/threonine kinase in the MAP kinase kinase kinase (MAP3K) family (MAP3K8) (Schlesinger et al. (1998) Front. Biosci. 3:D1181-86). Cot expression has been shown to induce ERK and JNK activation (Dumitru et al. (2000) Cell 103:1071-83; Chiariello et al. (2000) Mol. Cell. Biol. 20:1747-58). Similar to Raf, Cot is a MEK-1 kinase upstream of ERK pathway. Overexpression of Cot in CD3-activated T cells leads to IL-2 production, suggesting that Cot may play a role in T cell activation (Ballester et al. (1997) J. Immunol. 159:1613-18; Gilks et al. (1993) Mol. Cell. Biol. 13:1759-68). In addition, Cot has been shown to activate members of the NF-κB family, possibly by activating the IκB kinase (IKK) complex through NF-κB-inducing kinase (NIK) or by inducing the degradation of the inhibitory protein p105 (Kane et al. (2002) Mol. Cell. Biol. 22:5962-74; O'Mahony et al. (2000) Mol. Cell. Biol. 20:1170-78; Salmeron et al. (2001) J. Biol. Chem. 276:22215-22). Recent studies using Cot knockout mice point to a pivotal role of Cot in the LPS-induced production of TNF-α and other proinflammatory cytokines (Dumitru et al. (2000), supra; Eliopoulos et al. (2002) Embo. J. 21:4831-40).

Despite these various important cellular functions, the precise molecular mechanism of Cot regulation remains unsolved. As activation of both Cot and Raf lead to activation of downstream MAP kinase pathway components, and KSR is believed to function as a regulatory scaffold protein in the Raf/MEK/MAP kinase pathway, it would be desirable to evaluate the functional interrelationship of Cot and novel members of the KSR family.

### SUMMARY OF THE INVENTION

The invention provides novel isolated protein kinase polypeptides and the isolated nucleic acid molecules that encode them. The invention also provides genetically engineered expression vectors, host cells, and transgenic animals comprising the nucleic acid molecules of the invention. The invention additionally provides antisense and RNA interference (RNAi) molecules to the nucleic acid molecules of the invention. The invention further provides inhibitors, activators, and antibodies capable of binding to the protein kinase polypeptides of the invention. The invention also provides for the regulation of Cot/Tp12-mediated cellular functions, such as ERK and NF-κB activation, as well as IL-8 production, through the use of the nucleic acid and protein kinase polypeptide molecules of the invention.

### BRIEF DESCRIPTION OF THE DRAWINGS

FIG. 1 shows preferred siRNA molecules targeted to human KSR-2 mRNA for use in RNAi. Target segments [SEQ ID NOs:11-28; 65-93; 152-180; and 239-241] of the KSR-2 transcripts are grouped according to their first two nucleotides (AA, CA, GA, or TA) and are shown in the 5′−>3′ orientation. “GC Ratio” refers to the percentage of total G+C nucleotides in each target segment; “Position” refers to the nucleotide position in the human KSR-2 cDNA (SEQ ID NO:1) immediately preceding the beginning of each target segment. Preferred siRNA molecules (siRNA duplexes) are shown on the right side of the figure. The sense strand for each siRNA duplex [SEQ ID NOs:29-46; 94-122; 181-209; and 242-244] is shown in the 5′−>3′ orientation; the corresponding antisense strand [SEQ ID NOs:47-64; 123-151; 210-238; and 245-247] is shown in the 3′−>5′ orientation. For example, the siRNA molecule directed to the first target segment presented in the figure (i.e., SEQ ID NO:11) is the siRNA duplex of the sense and antisense strands identified (i.e., SEQ ID NO:29 and SEQ ID NO:47, respectively).

FIG. 2 shows a comparison (ClustalW multiple alignment) of the amino acid sequences of the KSR family members human KSR-2 (hKSR-2) (SEQ ID NO:2), mouse KSR-2 (mKSR-2) (SEQ ID NO:5), human KSR-1 (hKSR-1 [full-length (SEQ ID NO:250) and partial (SEQ ID NO:251)]), mouse KSR-1 (mKSR-1) (SEQ ID NO:252), Drosophila melanogaster KSR (DmKSR) (SEQ ID NO:253), and C. elegans KSR (CeKSR) (SEQ ID NO:254), and the positions of their predicted domains: the KSR-unique CA1 domain, the proline-rich CA2 domain, the cysteine-rich CA3 domain, the serine/threonine-rich CA4 domain, and the kinase catalytic CA5 domain.

### DETAILED DESCRIPTION OF THE INVENTION

The present invention provides novel isolated and purified polynucleotides and polypeptides homologous to KSR.

For example, the invention provides purified and isolated polynucleotides encoding a novel KSR (formerly referred to as N.KSR), herein designated “KSR-2.” Preferred DNA sequences of the invention include genomic and cDNA sequences and chemically synthesized DNA sequences.

The nucleotide sequence of a cDNA encoding this novel KSR, designated human KSR-2 cDNA, is set forth in SEQ ID NO:1. Polynucleotides of the present invention also include polynucleotides that hybridize under stringent conditions to SEQ ID NO:1, or its complement, and/or encode polypeptides that retain substantial biological activity of full-length human KSR-2. Polynucleotides of the present invention also include continuous portions of the sequence set forth in SEQ ID NO:1 comprising at least 21 consecutive nucleotides. A preferred polynucleotide of the present invention comprises the nucleotide sequence of SEQ ID NO:1 from nucleotide 1633 to nucleotide 2421.

The deduced amino acid sequence of human KSR-2 is set forth in SEQ ID NO:2. Polypeptides of the present invention also include continuous portions of the sequence set forth in SEQ ID NO:2 comprising at least seven consecutive amino acids. A preferred polypeptide of the present invention includes any continuous portion of the sequence set forth in SEQ ID NO:2 that retains substantial biological activity (i.e., an active fragment) of full-length human KSR-2. One such preferred polypeptide comprises the amino acid sequence of SEQ ID NO:2 from amino acid 545 to amino acid 807. Polynucleotides of the present invention also include, in addition to those polynucleotides of human origin described above, polynucleotides that encode the amino acid sequence set forth in SEQ ID NO:2 or a continuous portion thereof, and that differ from the polynucleotides of human origin described above due only to the well-known degeneracy of the genetic code.

The nucleotide sequence of a genomic DNA encoding this novel KSR, designated human KSR-2 genomic DNA, is set forth in SEQ ID NO:3. Polynucleotides of the present invention also include polynucleotides that hybridize under stringent conditions to SEQ ID NO:3, or its complement, and/or encode polypeptides that retain substantial biological activity of full-length human KSR-2. Polynucleotides of the present invention also include continuous portions of the sequence set forth in SEQ ID NO:3 comprising at least 21 consecutive nucleotides.

The nucleotide sequence of a cDNA encoding this novel KSR, designated mouse KSR-2 cDNA, is set forth in SEQ ID NO:4. Polynucleotides of the present invention also include polynucleotides that hybridize under stringent conditions to SEQ ID NO:4, or its complement, and/or encode polypeptides that retain substantial biological activity of full-length mouse KSR-2. Polynucleotides of the present invention also include continuous portions of the sequence set forth in SEQ ID NO:4 comprising at least 21 consecutive nucleotides. A preferred polynucleotide of the present invention comprises the nucleotide sequence of SEQ ID NO:4 from nucleotide 1636 to nucleotide 2424.

The deduced amino acid sequence of mouse KSR-2 is set forth in SEQ ID NO:5. Polypeptides of the present invention also include continuous portions of the sequence set forth in SEQ ID NO:5 comprising at least seven consecutive amino acids. A preferred polypeptide of the present invention includes any continuous portion of the sequence set forth in SEQ ID NO:5 that retains substantial biological activity (i.e., an active fragment) of full-length mouse KSR-2. One such preferred polypeptide comprises the amino acid sequence of SEQ ID NO:5 from amino acid 546 to amino acid 808. Polynucleotides of the present invention also include, in addition to those polynucleotides of murine origin described above, polynucleotides that encode the amino acid sequence set forth in SEQ ID NO:5 or a continuous portion thereof, and that differ from the polynucleotides of murine origin described above only due to the well-known degeneracy of the genetic code.

The nucleotide sequence of a genomic DNA encoding this novel KSR, designated mouse KSR-2 genomic DNA, is set forth in SEQ ID NO:6. Polynucleotides of the present invention also include polynucleotides that hybridize under stringent conditions to SEQ ID NO:6, or its complement, and/or encode polypeptides that retain substantial biological activity of full-length mouse KSR-2. Polynucleotides of the present invention also include continuous portions of the sequence set forth in SEQ ID NO:6 comprising at least 21 consecutive nucleotides.

The isolated polynucleotides of the present invention may be used as hybridization probes and primers to identify and isolate nucleic acids having sequences identical to or similar to those encoding the disclosed polynucleotides. Hybridization methods for identifying and isolating nucleic acids include polymerase chain reaction (PCR), Southern hybridizations, and Northern hybridization, and are well known to those skilled in the art.

Hybridization reactions can be performed under conditions of different stringency. The stringency of a hybridization reaction includes the difficulty with which any two nucleic acid molecules will hybridize to one another. Preferably, each hybridizing polynucleotide hybridizes to its corresponding polynucleotide under reduced stringency conditions, more preferably stringent conditions, and most preferably highly stringent conditions. Examples of stringency conditions are shown in Table 1 below: highly stringent conditions are those that are at least as stringent as, for example, conditions A-F; stringent conditions are at least as stringent as, for example, conditions G-L; and reduced stringency conditions are at least as stringent as, for example, conditions M-R.

| | | | Hybridization | |
|---|---|---|---|---|
| Stringency | Polynucleotide | Hybrid Length | Temperature and | Wash Temperature |
| Condition | Hybrid | (bp)¹ | Buffer² | and Buffer² |
| A | DNA:DNA | >50 | 65° C.; 1X SSC -or- | 65° C.; 0.3X SSC |
| | | | 42° C.; 1X SSC, | |
| | | | 50% formamide | |
| B | DNA:DNA | <50 | T_{B}*; 1X SSC | T_{B}*; 1X SSC |
| C | DNA:RNA | >50 | 67° C.; 1X SSC -or- | 67° C.; 0.3X SSC |
| | | | 45° C.; 1X SSC, | |
| | | | 50% formamide | |
| D | DNA:RNA | <50 | T_{D}*; 1X SSC | T_{D}*; 1X SSC |
| E | RNA:RNA | >50 | 70° C.; 1X SSC | 70° C.; 0.3X SSC |
| | | | -or- | |
| | | | 50° C.; 1X SSC, | |
| | | | 50% formamide | |
| F | RNA:RNA | <50 | T_{F}*; 1X SSC | T_{F}*; 1X SSC |
| G | DNA:DNA | >50 | 65° C.; 4X SSC | 65° C.; 1X SSC |
| | | | -or- | |
| | | | 42° C.; 4X SSC, | |
| | | | 50% formamide | |
| H | DNA:DNA | <50 | T_{H}*; 4X SSC | T_{H}*; 4X SSC |
| I | DNA:RNA | >50 | 67° C.; 4X SSC | 67° C.; 1X SSC |
| | | | -or- | |
| | | | 45° C.; 4X SSC | |
| | | | 50% formamide | |
| J | DNA:RNA | <50 | T_{J}*; 4X SSC | T_{J}*; 4X SSC |
| K | RNA:RNA | >50 | 70° C.; 4X SSC | 67° C.; 1X SSC |
| | | | -or- | |
| | | | 50° C.; 4X SSC, | |
| | | | 50% formamide | |
| L | RNA:RNA | <50 | T_{L}*; 2X SSC | T_{L}*; 2X SSC |
| M | DNA:DNA | >50 | 50° C.; 4X SSC | 50° C.; 2X SSC |
| | | | -or- | |
| | | | 40° C.; 6X SSC, | |
| | | | 50% formamide | |
| N | DNA:DNA | <50 | T_{N}*; 6X SSC | T_{N}*; 6X SSC |
| O | DNA:RNA | >50 | 55° C.; 4X SSC | 55° C.; 2X SSC |
| | | | -or- | |
| | | | 42° C.; 6X SSC, | |
| | | | 50% formamide | |
| P | DNA:RNA | <50 | T_{P}*; 6X SSC | T_{P}*; 6X SSC |
| Q | RNA:RNA | >50 | 60° C.; 4X SSC -or- | 60° C.; 2X SSC |
| | | | 45° C.; 6X SSC, | |
| | | | 50% formamide | |
| R | RNA:RNA | <50 | T_{R}*; 4X SSC | T_{R}*; 4X SSC |
| ¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known | | | | |
| # sequence are hybridized, the hybrid length can be determined by aligning the sequences of the polynucleotides and identifying the region or regions of optimal sequence complementarity. | | | | |
| ²SSPE (1xSSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers; washes are performed for 15 min after hybridization is complete. | | | | |
| T_{B}*-T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less | | | | |
| # than 18 base pairs in length, Tₘ(° C.) = 2(# of A + T bases) + 4(# of G + C bases). For hybrids between 18 and 49 base pairs in length, Tₘ(° C.) = 81.5 + 16.6(log₁₀Na⁺) + 0.41(%G⁺C) − (600/N), | | | | |
| # where N is the number of bases in the hybrid, and Na⁺ is the concentration of sodium ions in the hybridization buffer (Na⁺ for 1xSSC = 0.165M). | | | | |
| Additional examples of stringency conditions for polynucleotide hybridization are provided in Sambrook et al., Molecular Cloning: A Laboratory Manual, Chs. 9 & 11, Cold Spring Harbor Laboratory Press, Cold Spring Harbor, NY (1989), and Ausubel et al., eds., Current Protocols in Molecular Biology, | | | | |
| # Sects. 2.10 & 6.3-6.4, John Wiley & Sons, Inc. (1995), herein incorporated by reference. | | | | |

The isolated polynucleotides of the present invention may be used as hybridization probes and primers to identify and isolate DNAs having sequences encoding allelic variants of the disclosed polynucleotides. Allelic variants are naturally occurring alternative forms of the disclosed polynucleotides that encode polypeptides that are identical to or have significant similarity to the polypeptides encoded by the disclosed polynucleotides. Preferably, allelic variants have at least 90% sequence identity (more preferably, at least 95% identity; most preferably, at least 99% identity) with the disclosed polynucleotides.

The isolated polynucleotides of the present invention may also be used as hybridization probes and primers to identify and isolate DNAs having sequences encoding polypeptides homologous to the disclosed polynucleotides. These homologs are polynucleotides and polypeptides isolated from a different species than those of the disclosed polypeptides and polynucleotides, or within the same species, but with significant sequence similarity to the disclosed polynucleotides and polypeptides. Preferably, polynucleotide homologs have at least 60% sequence identity (more preferably, at least 75% identity; most preferably, at least 90% identity) with the disclosed polynucleotides, whereas polypeptide homologs have at least 30% sequence identity (more preferably, at least 45% identity; most preferably, at least 60% identity) with the disclosed polypeptides. Preferably, homologs of the disclosed polynucleotides and polypeptides are those isolated from mammalian species.

The isolated polynucleotides of the present invention may also be used as hybridization probes and primers to identify cells and tissues that express the polypeptides of the present invention and the conditions under which they are expressed.

Additionally, the isolated polynucleotides of the present invention may be used to alter (i.e., enhance, reduce, or modify) the expression of the genes corresponding to the polynucleotides of the present invention in a cell or an organism. These “corresponding genes” are the genomic DNA sequences of the present invention (e.g., SEQ ID NO:3 and SEQ ID NO:6) that are transcribed to produce the mRNAs from which the cDNA polynucleotides of the present invention (e.g., SEQ ID NO:1 and SEQ ID NO:4) are derived.

Altered expression of the genes of the present invention in a cell or organism may be achieved through the use of various inhibitory polynucleotides, such as antisense polynucleotides and ribozymes that bind and/or cleave the mRNA transcribed from the genes of the invention (e.g., Galderisi et al. (1999) J. Cell Physiol. 181:251-57; Sioud (2001) Curr. Mol. Med. 1:575-88).

The antisense polynucleotides or ribozymes of the invention can be complementary to an entire coding strand of a gene of the invention, or to a portion thereof. Alternatively, antisense polynucleotides or ribozymes can be complementary to a noncoding region of the coding strand of a gene of the invention. The antisense polynucleotides or ribozymes can be constructed using chemical synthesis and enzymatic ligation reactions using procedures well known in the art. The nucleoside linkages of chemically synthesized polynucleotides can be modified to enhance their ability to resist nuclease-mediated degradation, as well as to increase their sequence specificity. Such linkage modifications include, but are not limited to, phosphorothioate, methylphosphonate, phosphoroamidate, boranophosphate, morpholino, and peptide nucleic acid (PNA) linkages (Galderisi et al. (1999), supra; Heasman (2002) Dev. Biol. 243:209-14; Micklefield (2001) Curr. Med. Chem. 8:1157-79). Alternatively, these molecules can be produced biologically using an expression vector into which a polynucleotide of the present invention has been subcloned in an antisense (i.e., reverse) orientation.

The inhibitory polynucleotides of the present invention also include triplex-forming oligonucleotides (TFOs) which bind in the major groove of duplex DNA with high specificity and affinity (Knauert and Glazer (2001) Hum. Mol. Genet. 10:2243-51). Expression of the genes of the present invention can be inhibited by targeting TFOs complementary to the regulatory regions of the genes (i.e., the promoter and/or enhancer sequences) to form triple helical structures that prevent transcription of the genes.

In a preferred embodiment, the inhibitory polynucleotide of the present invention is a short interfering RNA (siRNA). siRNAs are short (preferably 19-25 nucleotides; most preferably 19 or 21 nucleotides), double-stranded RNA molecules that cause sequence-specific degradation of target mRNA. This degradation is known as RNA interference (RNAi) (e.g., Bass (2001) Nature 411:428-29). Originally identified in lower organisms, RNAi has been effectively applied to mammalian cells and has recently been shown to prevent fulminant hepatitis in mice treated with siRNAs targeted to Fas mRNA (Song et al. (2003) Nature Med. 9:347-51).

The siRNA molecules of the present invention can be generated by annealing two complementary single-stranded RNA molecules together (one of which matches a portion of the target mRNA) (Fire et al., U.S. Pat. No. 6,506,559) or through the use of a single hairpin RNA molecule which folds back on itself to produce the requisite double-stranded portion (Yu et al. (2002) Proc. Natl. Acad. Sci. USA 99:6047-52). The siRNA molecules can be chemically synthesized (Elbashir et al. (2001) Nature 411:494-98) or produced by in vitro transcription using single-stranded DNA templates (Yu et al. (2002), supra). Alternatively, the siRNA molecules can be produced biologically, either transiently (Yu et al. (2002), supra; Sui et al. (2002) Proc. Natl. Acad. Sci. USA 99:5515-20) or stably (Paddison et al. (2002) Proc. Natl. Acad. Sci. USA 99:1443-48), using an expression vector(s) containing the sense and antisense siRNA sequences. Recently, reduction of levels of target mRNA in primary human cells, in an efficient and sequence-specific manner, was demonstrated using adenoviral vectors that express hairpin RNAs, which are further processed into siRNAs (Arts et al. (2003) Genome Res. 13:2325-32).

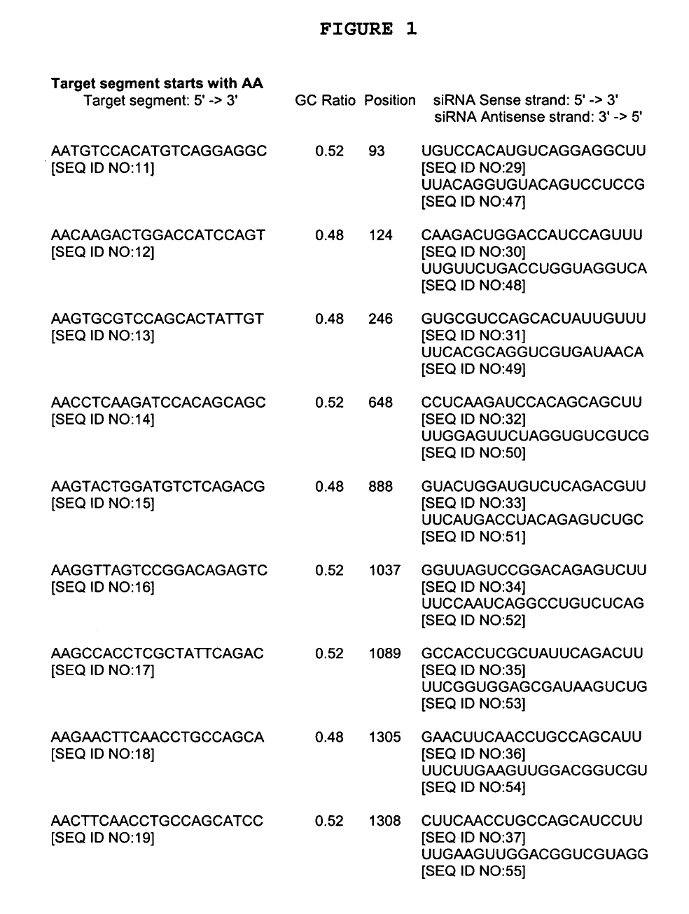

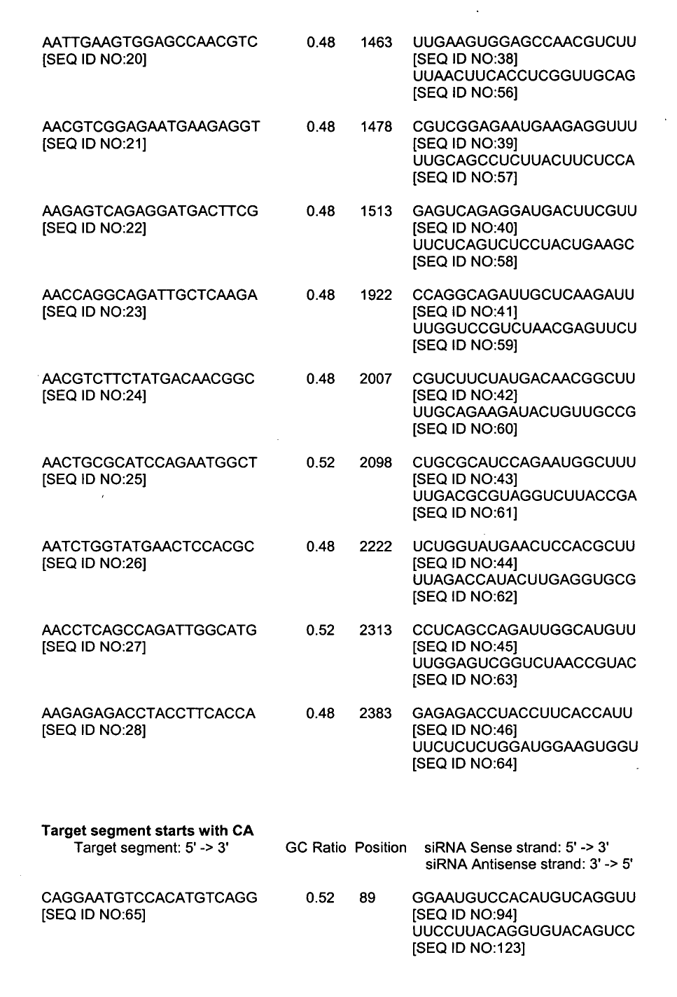

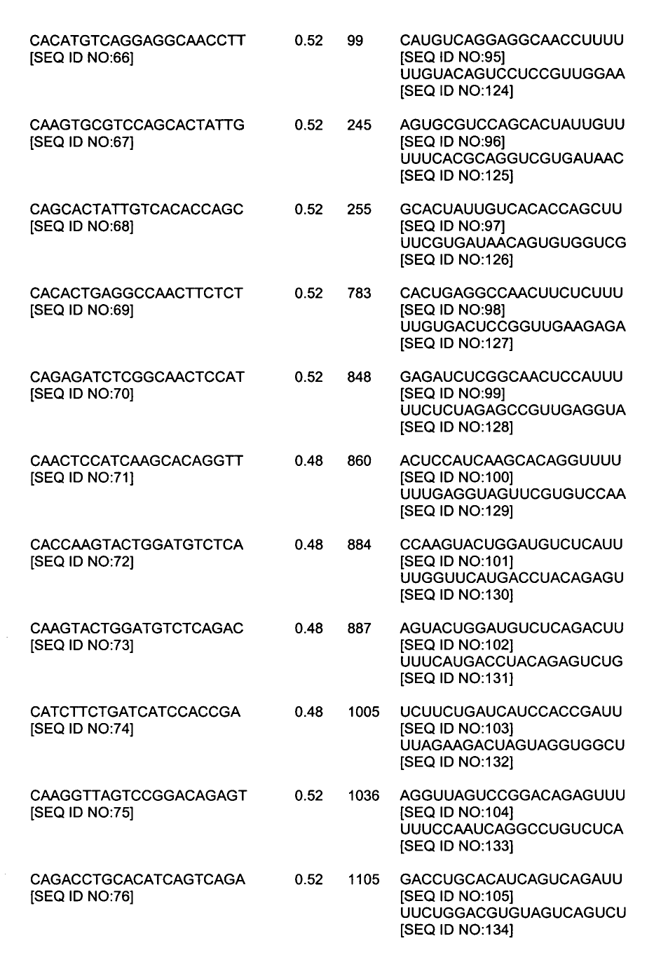

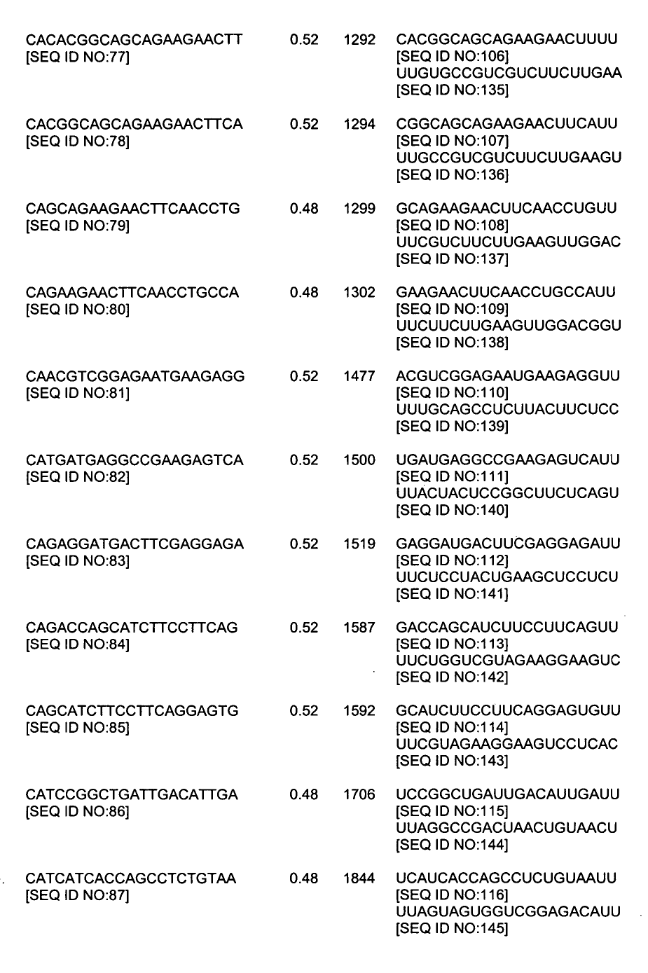

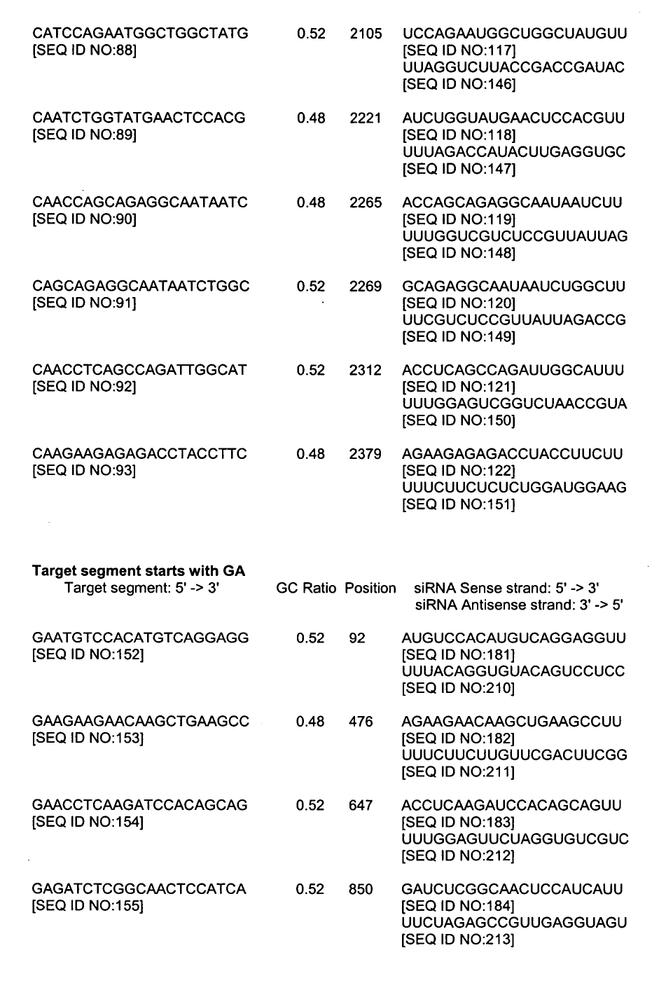

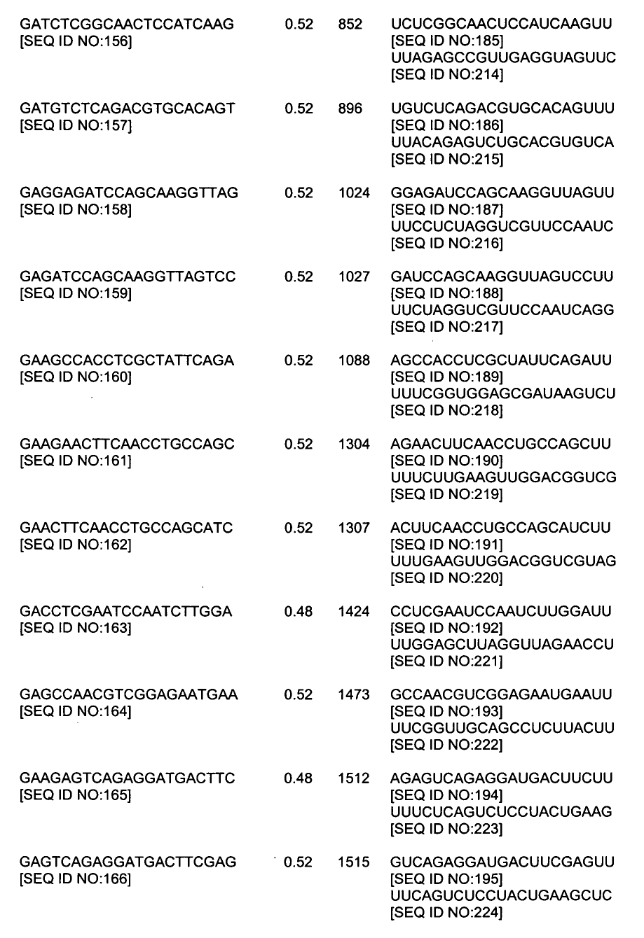

The siRNA molecules targeted to the polynucleotides of the present invention can be designed based on criteria well known in the art (e.g., Elbashir et al. (2001) EMBO J., 20:6877-88). For example, the target segment of the target mRNA preferably should begin with AA (most preferred), TA, GA, or CA; the GC ratio of the siRNA molecule preferably should be 45-55%; the siRNA molecule preferably should not contain three of the same nucleotides in a row; the siRNA molecule preferably should not contain seven mixed G/Cs in a row; and the target segment preferably should be in the ORF region of the target mRNA and preferably should be at least 75 bp after the initiation ATG and at least 75 bp before the stop codon. Based on these criteria, preferred siRNA molecules of the present invention have been designed and are shown in FIG. 1. Other siRNA molecules targeted to the polynucleotides of the present invention can be designed by one of ordinary skill in the art using the aforementioned criteria or other known criteria (e.g., Reynolds et al. (2004) Nature Biotechnol. 22:326-30).

Altered expression of the genes of the present invention in an organism may also be achieved through the creation of nonhuman transgenic animals into whose genomes polynucleotides of the present invention have been introduced. Such transgenic animals include animals that have multiple copies of a gene (i.e., the transgene) of the present invention. A tissue-specific regulatory sequence(s) may be operably linked to the transgene to direct expression of a polypeptide of the present invention to particular cells or a particular developmental stage. Methods for generating transgenic animals via embryo manipulation and microinjection, particularly animals such as mice, have become conventional and are well known in the art (e.g., Bockamp et al. (2002) Physiol. Genomics 11:115-32).

Altered expression of the genes of the present invention in an organism may also be achieved through the creation of animals whose endogenous genes corresponding to the polynucleotides of the present invention have been disrupted through insertion of extraneous polynucleotide sequences (i.e., a knockout animal). The coding region of the endogenous gene may be disrupted, thereby generating a nonfunctional protein. Alternatively, the upstream regulatory region of the endogenous gene may be disrupted or replaced with different regulatory elements, resulting in the altered expression of the still-functional protein. Methods for generating knockout animals include homologous recombination and are well known in the art (e.g., Wolfer et al. (2002) Trends Neurosci. 25:336-40).

The isolated polynucleotides of the present invention may be operably linked to an expression control sequence for recombinant production of the polypeptides of the present invention. General methods of expressing recombinant proteins are well known in the art.

A number of cell lines may act as suitable host cells for recombinant expression of the polypeptides of the present invention. Mammalian host cell lines include, e.g., COS cells, CHO cells, 293T cells, A431 cells, 3T3 cells, CV-1 cells, HeLa cells, L cells, BHK21 cells, HL-60 cells, U937 cells, HaK cells, Jurkat cells, as well as cell strains derived from in vitro culture of primary tissue and primary explants.

Alternatively, it may be possible to recombinantly produce the polypeptides of the present invention in lower eukaryotes such as yeast or in prokaryotes. Potentially suitable yeast strains include Saccharomyces cerevisiae, Schizosaccharomyces pombe, Kluyveromyces strains, and Candida strains. Potentially suitable bacterial strains include Escherichia coli, Bacillus subtilis, and Salmonella typhimurium. If the polypeptides of the present invention are made in yeast or bacteria, it may be necessary to modify them by, for example, phosphorylation or glycosylation of appropriate sites, in order to obtain functionality. Such covalent attachments may be accomplished using well-known chemical or enzymatic methods.

The polypeptides of the present invention may also be recombinantly produced by operably linking the isolated polynucleotides of the present invention to suitable control sequences in one or more insect expression vectors, such as baculovirus vectors, and employing an insect cell expression system. Materials and methods for baculovirus/Sf9 expression systems are commercially available in kit form (e.g., the MaxBac® kit, Invitrogen, Carlsbad, Calif.).

Following recombinant expression in the appropriate host cells, the polypeptides of the present invention may then be purified from culture medium or cell extracts using known purification processes, such as gel filtration and ion exchange chromatography. Purification may also include affinity chromatography with agents known to bind the polypeptides of the present invention. These purification processes may also be used to purify the polypeptides of the present invention from natural sources.

Alternatively, the polypeptides of the present invention may also be recombinantly expressed in a form that facilitates purification. For example, the polypeptides may be expressed as fusions with proteins such as maltose-binding protein (MBP), glutathione-S-transferase (GST), or thioredoxin (TRX). Kits for expression and purification of such fusion proteins are commercially available from New England BioLabs (Beverly, Mass.), Pharmacia (Piscataway, N.J.), and Invitrogen, respectively. The polypeptides of the present invention can also be tagged with a small epitope and subsequently identified or purified using a specific antibody to the epitope. A preferred epitope is the FLAG epitope, which is commercially available from Eastman Kodak (New Haven, Conn.).

The polypeptides of the present invention may also be produced by known conventional chemical synthesis. Methods for chemically synthesizing the polypeptides of the present invention are well known to those skilled in the art. Such chemically synthetic polypeptides may possess biological properties in common with the natural, purified polypeptides, and thus may be employed as biologically active or immunological substitutes for the natural polypeptides.

The polypeptides of the present invention also encompass molecules that are structurally different from the disclosed polypeptides (e.g., which have a slightly altered sequence), but which have substantially the same biochemical properties as the disclosed polypeptides (e.g., are changed only in functionally nonessential amino acid residues). Such molecules include naturally occurring allelic variants and deliberately engineered variants containing alterations, substitutions, replacements, insertions, or deletions. Techniques for such alterations, substitutions, replacements, insertions, or deletions are well known to those skilled in the art.

Antibody molecules to the polypeptides of the present invention may be produced by methods well known to those skilled in the art. For example, monoclonal antibodies can be produced by generation of hybridomas in accordance with known methods. Hybridomas formed in this manner are then screened using standard methods, such as enzyme-linked immunosorbent assay (ELISA), to identify one or more hybridomas that produce an antibody that specifically binds with the polypeptides of the present invention. A full-length polypeptide of the present invention may be used as the immunogen, or, alternatively, antigenic peptide fragments of the polypeptides may be used. An antigenic peptide of a polypeptide of the present invention comprises at least seven continuous amino acid residues and encompasses an epitope such that an antibody raised against the peptide forms a specific immune complex with the polypeptide. Preferably, the antigenic peptide comprises at least 10 amino acid residues, more preferably at least 15 amino acid residues, even more preferably at least 20 amino acid residues, and most preferably at least 30 amino acid residues.

As an alternative to preparing monoclonal antibody-secreting hybridomas, a monoclonal antibody to a polypeptide of the present invention may be identified and isolated by screening a recombinant combinatorial immunoglobulin library (e.g., an antibody phage display library) with a polypeptide of the present invention to thereby isolate immunoglobulin library members that bind to the polypeptide. Techniques and commercially available kits for generating and screening phage display libraries are well known to those skilled in the art. Additionally, examples of methods and reagents particularly amenable for use in generating and screening antibody display libraries can be found in the literature.

Polyclonal sera and antibodies may be produced by immunizing a suitable subject with a polypeptide of the present invention. The antibody titer in the immunized subject may be monitored over time by standard techniques, such as with ELISA using immobilized marker protein. If desired, the antibody molecules directed against a polypeptide of the present invention may be isolated from the subject or culture media and further purified by well-known techniques, such as protein A chromatography, to obtain an IgG fraction.

Fragments of antibodies to the polypeptides of the present invention may be produced by cleavage of the antibodies in accordance with methods well known in the art. For example, immunologically active F(ab′) and F(ab′)₂ fragments may be generated by treating the antibodies with an enzyme such as pepsin.

Additionally, chimeric, humanized, and single-chain antibodies to the polypeptides of the present invention, comprising both human and nonhuman portions, may be produced using standard recombinant DNA techniques. Humanized antibodies may also be produced using transgenic mice which are incapable of expressing endogenous immunoglobulin heavy and light chain genes, but which can express human heavy and light chain genes.

The polynucleotides and polypeptides of the present invention may also be used in screening assays to identify pharmacological agents or lead compounds for agents capable of modulating KSR-2 activity, either substrate binding and/or kinase activity. Such screening assays are well known in the art (e.g., Xing et al. (2000) J. Biol. Chem. 275:17276-80; Turek et al. (2001) Anal. Biochem. 299:45-53). For example, samples containing KSR-2 (either natural or recombinant) can be contacted with one of a plurality of test compounds (either small organic molecules or biological agents), and the activity of KSR-2 in each of the treated samples can be compared to the activity of KSR-2 in untreated samples or in samples contacted with different test compounds to determine whether any of the test compounds provides: 1) a substantially decreased level of KSR-2 activity, thereby indicating an inhibitor of KSR-2 activity, or 2) a substantially increased level of KSR-2 activity, thereby indicating an activator of KSR-2 activity. In a preferred embodiment, the identification of test compounds capable of modulating KSR-2 activity is performed using high-throughput screening assays, such as provided by BIACORE® (Biacore International AB, Uppsala, Sweden), BRET (bioluminescence resonance energy transfer), and FRET (fluorescence resonance energy transfer) assays, as well as ELISA.

The present invention is illustrated by the following Examples related to human and mouse cDNAs, designated human KSR-2 cDNA and mouse KSR-2 cDNA, respectively, encoding novel KSR polypeptides designated KSR-2.

### EXAMPLES

The Examples which follow are set forth to aid in the understanding of the invention but are not intended to, and should not be construed to, limit its scope in any way. The Examples do not include detailed descriptions of conventional methods, such as those employed in the construction of vectors and plasmids, the insertion of genes encoding polypeptides into such vectors and plasmids, the introduction of such vectors and plasmids into host cells, or the expression of polypeptides from such vectors and plasmids in host cells. Such methods are well known to those of ordinary skill in the art and are described in numerous publications.

### Example 1

Identification of KSR-2 DNA Sequences

### Example 1.1

### Identification of the Human KSR-2 Genomic and cDNA Sequence

Human KSR-2 was initially predicted by structural-based genome data mining using novel computational techniques based on bioinformatics principles described in Baxevanis and Ouellette, eds., Bioinformatics: A Practical Guide to the Analysis of Genes and Proteins, Wiley InterScience, New York (2001), herein incorporated by reference in its entirety. The hKSR-2 cDNA was derived from genomic database mining using the structural profiles of the catalytic domains of various kinases. To achieve this, all available X-ray crystal structures of the catalytic domains of serine/threonine and tyrosine protein kinases were collected from the SCOP database (http://scop.berkeley.edu). Structural alignments were performed using the ProCeryon package (http://www.proceryon.com). This alignment was converted into a “scoring matrix” which carries the structural profile of the kinase catalytic domains. The structural alignments were presented in the form of multiple sequence alignments, which were used to search the Celera Human Genome Genscan predictions (release R25h) using NCBI's psiblast package. DNA sequences encoding the predicted protein sequences aligning to the scoring matrix were extracted from public human genomic sequence databases, such as Celera and GenBank. The extracted nucleic acid sequences were clustered to eliminate repetitive entries. The putative kinase domains were then sequentially run through a series of queries and filters to identify novel protein kinase sequences. Specifically, the identified sequences were used to search a nucleotide and amino acid repository of known human protein kinases using BLASTN and BLASTX. The output was parsed into a spreadsheet to facilitate elimination of known genes by manual inspection. The selected hits were then queried using BLASTN against the NCBI nr and est databases to confirm their novelty and also to select the closest homologs.

Extensions of the partial DNA sequences were performed using the Genewise program to predict potential open reading frames based on homology to the closest homologs. Genewise requires two input sequences: the homologous protein and the genomic DNA containing the gene of interest. The homologs were identified by BLASTP searches of the NCBI nr protein database with the novel kinase hits described above. The genomic DNA was identified by BLASTN searches of the Celera and GenBank databases. The extended “virtual” cDNA sequences were then used to isolate the corresponding physical clones from cDNA libraries.

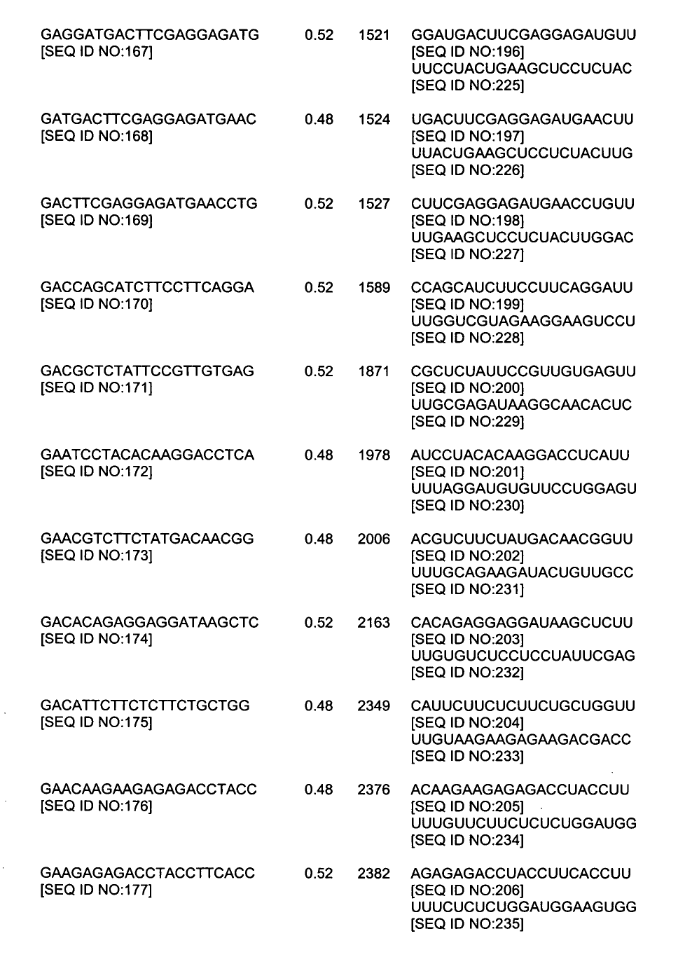

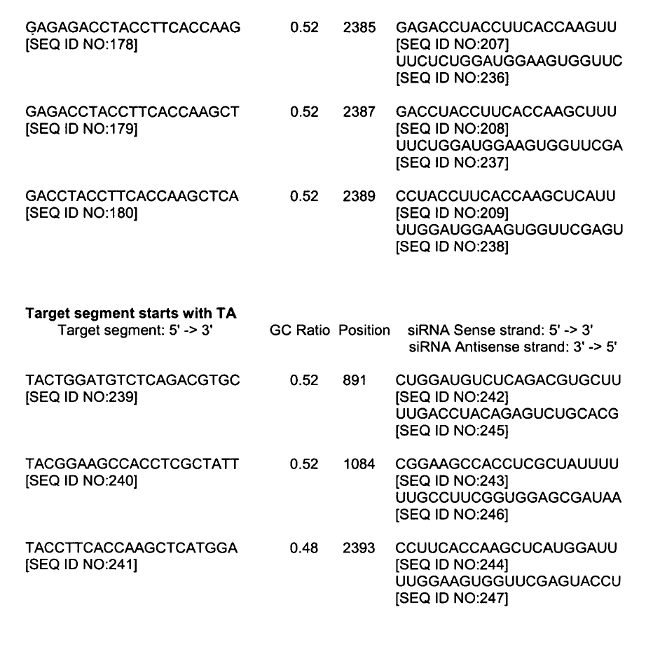

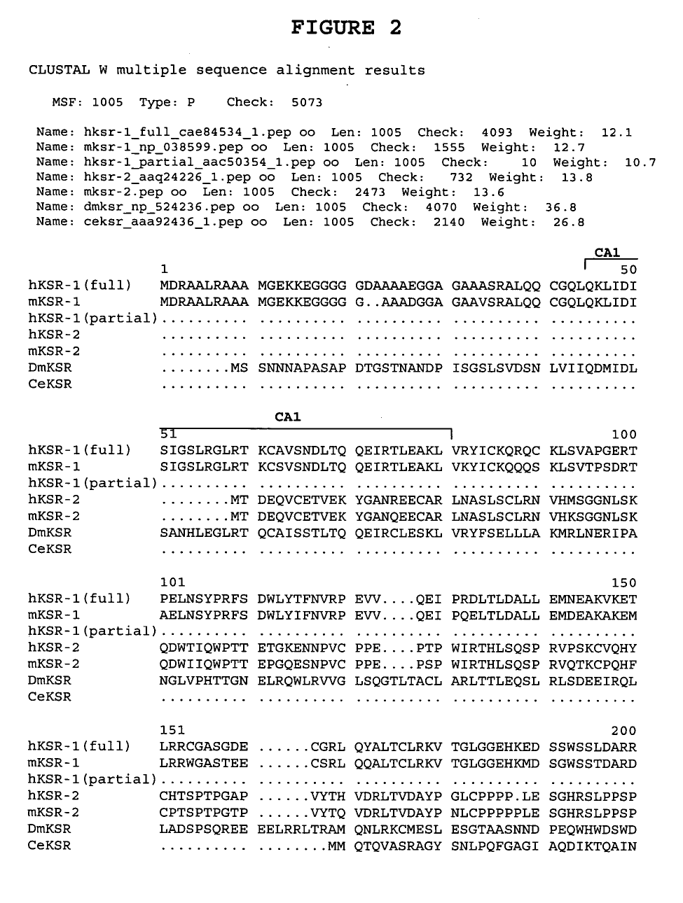

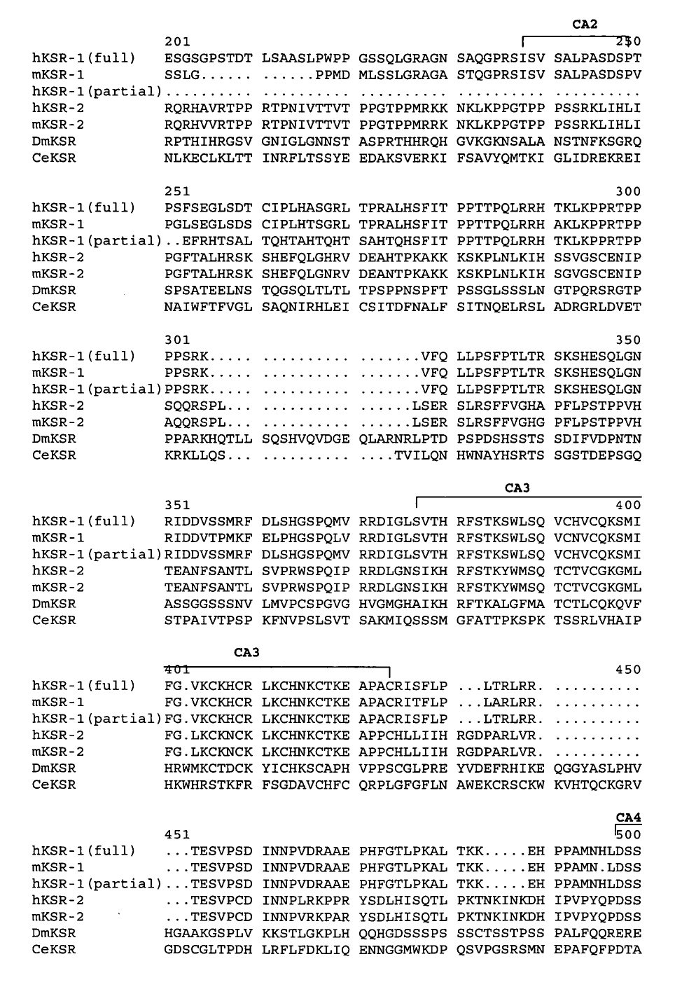

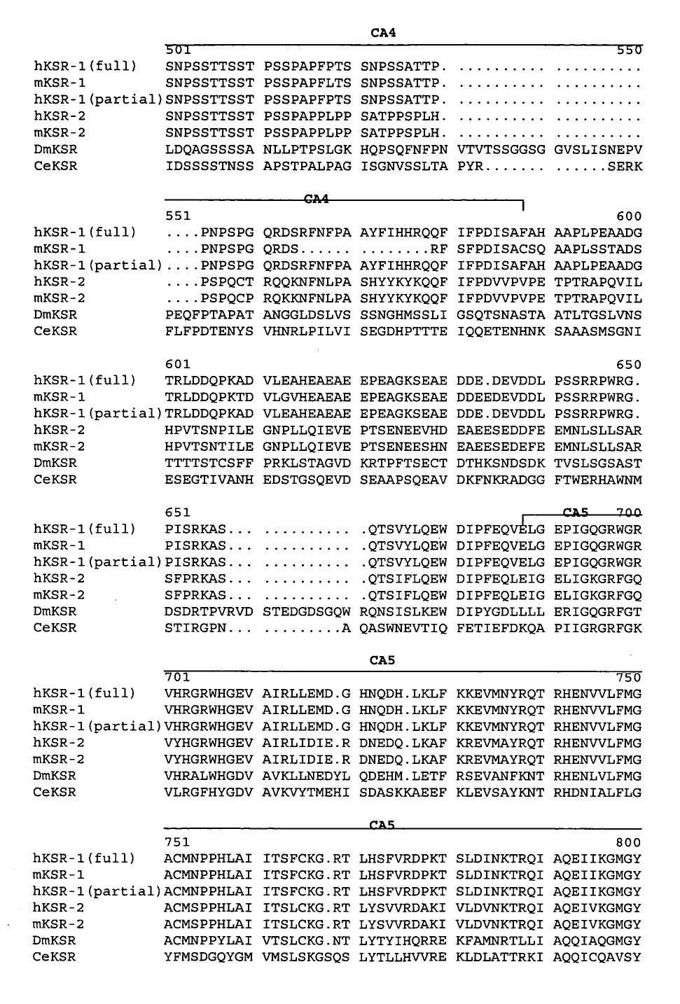

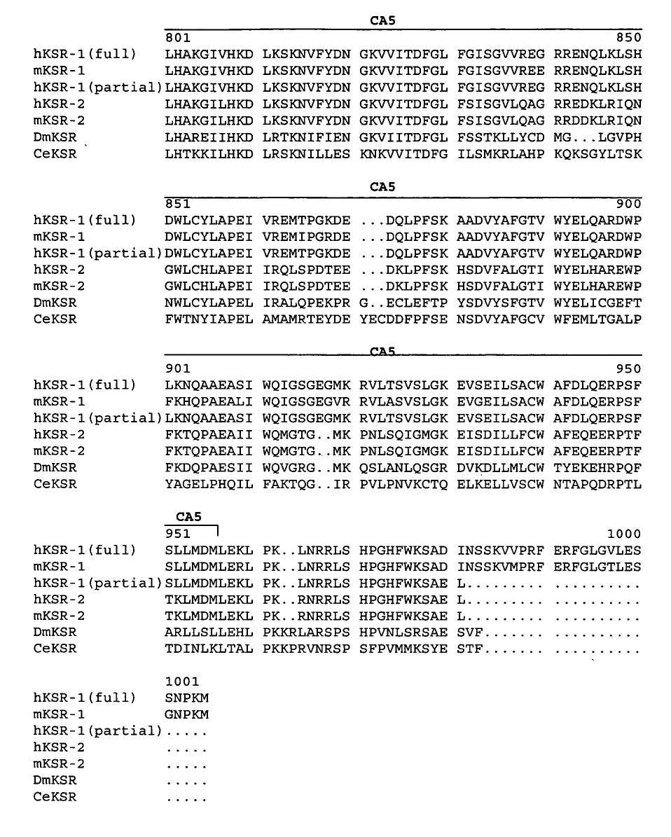

Based on such a virtual cDNA sequence, a predicted gene, which was homologous to the known human KSR, was identified. This predicted sequence was supported by an expressed sequence tag (EST) clone (BF948353). The EST-supported region was selected as a gene capture oligo for cloning, and two isolates (B11 and F7) were obtained from a human testis cDNA library. The presence of an in-frame stop codon upstream of the predicted initiator methionine argues that this cDNA contains the complete open reading frame (ORF). Based on the multiple sequence alignments of known KSR family members, the N-terminus of B11 and C-terminus of F7 were combined by PCR to obtain a novel cDNA. The novel KSR-like clone was termed human KSR-2. The human KSR-2 cDNA sequence, and its deduced amino acid sequence, are set forth in SEQ ID NOs:1 and 2, respectively. The hKSR-2 cDNA has an ORF of 2487 bp (coding sequence of 2490 bp) that encodes an 829 aa protein kinase. hKSR-2 contains proline-rich CA2, cysteine-rich CA3, serine/threonine repeats and the consensus MAPK phosphorylation site of CA4, and the conserved kinase catalytic domain CA5 (FIG. 2). However, hKSR-2 lacks subdomain CA1, which is a domain unique to the KSR family (Therrien et al. (1995), supra). A murine homolog of hKSR-2 (mKSR-2) was predicted using Genewise software and a cDNA clone was obtained from a mouse kidney cDNA library. The 5893-bp mouse cDNA has 88.9% identity to the hKSR-2 cDNA, and the predicted 830-aa protein has 96.9% identity at the amino acid level with hKSR-2. Northern blot revealed that hKSR-2 is mainly expressed in brain and kidney.

BLASTP analysis with the deduced amino acid sequence of human KSR-2 (SEQ ID NO:2) revealed significant homology to the kinase domains of KSR from various species, including human KSR-1 (partial—GenBank Acc. No. U43586; and full-length—GenBank Acc. No. CAE84534_{—}1), mouse KSR-1 (GenBank Acc. No. U43585), Drosophila melanogaster KSR (GenBank Acc. No. U43583), Drosophila virilis KSR (GenBank Acc. No. U43584), Drosophila simulans KSR (GenBank Acc. No. AY135139), and C. elegans KSR-1 (GenBank Acc. No. U38820). ClustalW alignment of the deduced amino acid sequences of human KSR-2 and human and mouse KSR-1 (FIG. 2) revealed that human KSR-2, like human KSR-1, contains the proline-rich CA2 domain, the cysteine-rich CA3 domain, the serine/threonine-rich CA4 domain, and the kinase catalytic CA5 domain characteristic of all known KSR proteins (Morrison, supra). Interestingly, KSR-2 lacks the CA1 domain unique to known KSR proteins.

Comparisons related to the genomic DNA used for the Genewise extension (described above) revealed that the human KSR-2 locus, which maps to human chromosome 12q24.3 (position numbers 117539035-117930464, according to Celera mapping), contains 18 exons and 17 introns (see Table 2, below). The human KSR-2 genomic DNA sequence is set forth in SEQ ID NO:3.

TABLE 2

| Region in | | | Position in |
|---|---|---|---|
| SEQ ID NO: 3 | Sequence Attribute | Length (bp) | SEQ ID NO: 1 |
| 1-2000 | 5′-sequence¹ | 2000 | — |
| 2001-2109 | Exon#1 | 109 | 1-109 |
| 2110-95789 | Intron#1 | 93680 | — |
| 95790-96303 | Exon#2 | 514 | 110-623 |
| 96304-189629 | Intron#2 | 93326 | — |
| 189630-189814 | Exon#3 | 185 | 624-808 |
| 189815-274910 | Intron#3 | 85096 | — |
| 274911-274980 | Exon#4 | 70 | 809-878 |
| 274981-278079 | Intron#4 | 3099 | — |
| 278080-278163 | Exon#5 | 84 | 879-962 |
| 278164-298713 | Intron#5 | 20550 | — |
| 298714-298781 | Exon#6 | 68 | 963-1030 |
| 298782-301993 | Intron#6 | 3212 | — |
| 301994-302118 | Exon#7 | 125 | 1031-1155 |
| 302119-317400 | Intron#7 | 15282 | — |
| 317401-317569 | Exon#8 | 169 | 1156-1324 |
| 317570-325580 | Intron#8 | 8011 | — |
| 325581-325622 | Exon#9 | 42 | 1325-1366 |
| 325623-326274 | Intron#9 | 652 | — |
| 326275-326347 | Exon#10 | 73 | 1367-1439 |
| 326348-330168 | Intron#10 | 3821 | — |
| 330169-330217 | Exon#11 | 49 | 1440-1488 |
| 330218-332068 | Intron#11 | 1851 | — |
| 332069-332436 | Exon#12 | 368 | 1489-1856 |
| 332437-371597 | Intron#12 | 39161 | — |
| 371598-371694 | Exon#13 | 97 | 1857-1953 |
| 371695-372739 | Intron#13 | 1045 | — |
| 372740-372873 | Exon#14 | 134 | 1954-2087 |
| 372874-380695 | Intron#14 | 7822 | — |
| 380696-380827 | Exon#15 | 132 | 2088-2219 |
| 380828-385970 | Intron#15 | 5143 | — |
| 385971-386100 | Exon#16 | 130 | 2220-2349 |
| 386101-387495 | Intron#16 | 1395 | — |
| 387496-387629 | Exon#17 | 134 | 2350-2483 |
| 387630-390085 | Intron#17 | 2456 | — |
| 390086-390089 | Exon#18 | 4 | 2484-2487 |
| 390090-390092 | Stop | 3 | 2488-2890 |
| 390093-392092 | 3′-sequence² | 2000 | — |

¹5′-sequence includes 5′-UTR (untranslated region) and/or genomic sequences
²3′-sequence includes 3′-UTR (untranslated region) and/or genomic sequences

A search of public single nucleotide polymorphism (SNP) databases revealed that the human KSR-2 locus contains 755 SNPs, only two of which occur in the coding region of human KSR-2 (a C/G transversion at nucleotide position 95833 of SEQ ID NO:3 in exon 2, and an A/G transition at nucleotide position 302028 of SEQ ID NO:3 in exon 7).

### Example 1.2

### Identification of the Mouse KSR-2 cDNA Sequence

A mouse KSR-2 virtual cDNA was extracted from public murine genomic DNA databases based on the human KSR-2 cDNA and mouse KSR-1 cDNA (GenBank Acc. No. U43585) sequences. This virtual cDNA sequence was used to isolate a physical clone from a mouse kidney library with an open reading frame of 2490 bp (coding sequence of 2493 bp). This mouse KSR-2 cDNA sequence is set forth in SEQ ID NO:4. The deduced amino acid sequence of mouse KSR-2 is set forth in SEQ ID NO:5.

Like human KSR-2, BLASTP analysis with the deduced amino acid sequence of mouse KSR-2 (SEQ ID NO:5) revealed significant homology to the kinase domains of KSR from various species, including human KSR-1 (GenBank Acc. No. U43586), mouse KSR-1 (GenBank Acc. No. U43585), D. melanogaster KSR (GenBank Acc. No. U43583), D. virilis KSR (GenBank Acc. No. U43584), D. simulans KSR (GenBank Acc. No. AY135139), and C. elegans KSR-1 (GenBank Acc. No. U38820). ClustalW alignment revealed that, like human KSR-2, mouse KSR-2 contains the proline-rich CA2 domain, the cysteine-rich CA3 domain, the serine/threonine-rich CA4 domain, and the kinase catalytic CA5 domain, but lacks the CA1 domain (FIG. 2).

Comparison of the mouse KSR-2 cDNA sequence with the genomic DNA used to extract the virtual cDNA described above revealed that the mouse KSR-2 locus, which maps to mouse chromosome 5F (position numbers 111593985-111857837, according to Celera mapping), contains 18 exons and 17 introns (see Table 3, below). A comparison of Table 2 and Table 3 shows that exonic size and intronic position are well conserved between the human and mouse KSR-2 genomic DNA sequences. The mouse KSR-2 genomic DNA sequence is set forth in SEQ ID NO:6.

TABLE 3

| Region in | | | Position in |
|---|---|---|---|
| SEQ ID NO: 6 | Sequence Attribute | Length (bp) | SEQ ID NO: 4 |
| 1-2000 | 5′-sequence¹ | 2000 | — |
| 2001-2109 | Exon#1 | 109 | 1-109 |
| 2110-51728 | Intron#1 | 49619 | — |
| 51729-52234 | Exon#2 | 506 | 110-626 |
| 52235-113307 | Intron#2 | 61073 | — |
| 113308-113492 | Exon#3 | 185 | 627-811 |
| 113493-164953 | Intron#3 | 51461 | — |
| 164954-165023 | Exon#4 | 70 | 812-881 |
| 165024-168073 | Intron#4 | 3050 | — |
| 168074-168157 | Exon#5 | 84 | 882-965 |
| 168158-181058 | Intron#5 | 12901 | — |
| 181059-181126 | Exon#6 | 68 | 966-1033 |
| 181127-185150 | Intron#6 | 4024 | — |
| 185151-185275 | Exon#7 | 125 | 1034-1158 |
| 185276-190967 | Intron#7 | 5692 | — |
| 190968-191136 | Exon#8 | 169 | 1159-1327 |
| 191137-198340 | Intron#8 | 7204 | — |
| 198341-198382 | Exon#9 | 42 | 1328-1369 |
| 198383-198850 | Intron#9 | 468 | — |
| 198851-198923 | Exon#10 | 73 | 1370-1442 |
| 198924-202512 | Intron#10 | 3589 | — |
| 202513-202561 | Exon#11 | 49 | 1443-1491 |
| 202562-203758 | Intron#11 | 1197 | — |
| 203759-204126 | Exon#12 | 368 | 1492-1859 |
| 204127-243160 | Intron#12 | 39034 | — |
| 243161-243257 | Exon#13 | 97 | 1860-1956 |
| 243258-244905 | Intron#13 | 1648 | — |
| 244906-245039 | Exon#14 | 134 | 1957-2090 |
| 245040-252539 | Intron#14 | 7500 | — |
| 252540-252671 | Exon#15 | 132 | 2091-2222 |
| 252672-257603 | Intron#15 | 4932 | — |
| 257604-257733 | Exon#16 | 130 | 2223-2352 |
| 257734-259474 | Intron#16 | 1741 | — |
| 259475-259608 | Exon#17 | 134 | 2353-2486 |
| 259609-261846 | Intron#17 | 2238 | — |
| 261847-261850 | Exon#18 | 4 | 2487-2490 |
| 261851-261853 | Stop | 3 | 2491-2493 |
| 261854-263853 | 3′-sequence² | 2000 | — |

¹5′-sequence includes 5′-UTR (untranslated region) and/or genomic sequences
²3′-sequence includes 3′-UTR (untranslated region) and/or genomic sequences

A search of public SNP databases revealed that the mouse KSR-2 locus contains 287 SNPs, only one of which occurs in the coding region of mouse KSR-2 (a T/C transition at nucleotide position 113353 of SEQ ID NO:6 in exon 3).

### Example 2

Tissue Expression of the Human KSR-2 Gene

### Example 2.1

### Northern Analysis

Tissue expression of human KSR-2 was first assessed by Northern analysis using a Clontech Multiple Tissue Northern (MTN) Blot (Palo Alto, Calif.). MTN blots contain approximately 1 μg of polyA⁺ RNA/lane from twelve human tissues. The RNA was run on a denaturing formaldehyde 1.0% agarose gel, transferred to a nylon membrane, and fixed by UV irradiation.

Based upon the cDNA sequence of human KSR-2 (SEQ ID NO:1), PCR primers were designed to amplify a 333 bp fragment (SEQ ID NO:7) from the human KSR-2 cDNA using PCR. The primers were designed to amplify coding sequence corresponding to exons 8-11 of the human KSR-2 genomic DNA sequence (SEQ ID NO:3), thereby avoiding the highly conserved kinase domain and reducing the possibility of nonspecific hybridization. The sequences of the forward (SEQ ID NO:8) and reverse (SEQ ID NO:9) primers were:

| | |
|---|---|
| 5′ GACCACATCCCTGTCCCTTA 3′ | (forward primer) |
| 5′ CTCCGACGTTGGCTCCACTT 3′ | (reverse primer) |

The amplified fragment was gel purified and sequence confirmed. The fragment was labeled with [α³²P]dCTP by random priming to produce the Northern probe.

The MTN blot was hybridized with 1-2×10⁶ cpm/mL ³²P probe in QuickHyb™ buffer (Stratagene, La Jolla, Calif.) along with 150 μg denatured sonicated salmon sperm DNA at 68° C. for 2-4 h. The blot was washed with 2×SSC/1% SDS and 0.1×SSC/1% SDS multiple times at 65° C. Following the washes, the blot was exposed to film for multiple exposures. Smeary high molecular weight bands, which were inconclusive as to the size of the transcripts, were identified mainly in brain and kidney.

### Example 2.2

### Tissue Array Analysis

The tissue expression of KSR-2 was further analyzed using a Clontech Multiple Tissue Expression (MTE) array. MTE arrays are dot blots containing normalized loadings of polyA⁺ RNA from 72 different human tissues and eight different control RNAs and DNAs.

The MTE blot was hybridized with 1-2×10⁶ cpm/mL ³²P probe (as described above) in QuickHyb™ buffer along with 150 μg denatured sonicated salmon sperm DNA and 30 μg denatured human Cₒt-1 DNA at 65° C. for approximately 18 h. The blot was washed with 2×SSC/1% SDS and 0.1×SSC/1% SDS multiple times at 65° C. Following the washes, the blot was exposed to film for multiple exposures. The most prominent expression of human KSR-2 was detected in various brain regions, with weaker expression in the lymph node, testis, kidney, and adrenal gland.

### Example 2.3

### Cancer Array Analysis

The expression of human KSR-2 in various human cancers was assessed using a Clontech Cancer Profiling Array (CPA). CPAs are dot blots of 241 paired cDNA samples from tumor and adjacent normal tissue from individual patients.

The CPA blot was hybridized with 1-2×10⁶ cpm/mL ³²P probe (as described above) in ExpressHyb™ buffer (Clontech) along with 150 μg denatured sonicated salmon sperm DNA and 30 μg denatured human Cₒt-1 DNA at 65° C. for approximately 18 h. The blot was washed with 2×SSC/1% SDS and 0.1×SSC/1% SDS multiple times at 65° C. Following the washes, the blot was exposed to film for multiple exposures. No obvious differential expression of KSR-2 between normal and tumor tissues was identified.

### Example 3

Recombinant Expression and Biochemical Analysis of KSR-2

### Example 3.1

### Recombinant Expression of Human KSR-2

Human KSR-2 was initially predicted by whole genomic data mining using the structural profile of the catalytic domains of various protein kinases (as described above). To isolate the human KSR-2 physical clone, an EST (GenBank Acc. No. BF948353) -supported region of the gene (spanning nucleotides 1095-1324 of SEQ ID NO:1) was used as a gene capture oligonucleotide for cloning. Two overlapping partial cDNA clones (F7 and B11) were isolated from a human testis Marathon-Ready cDNA library (Clontech). The full-length KSR-2 was generated by combining the two partial cDNA clones in a PCR reaction. The resulting full-length sequence was cloned into pENTR/SD-TOPO (Invitrogen). The FLAG epitope, DYKDDDDK (SEQ ID NO:10), was incorporated at the 3′ end of KSR-2 by PCR. The resulting product, termed hKSR-2-FLAG, was cloned into pcDNA-DEST-40 (Invitrogen) for mammalian expression. Similarly, the catalytic domain of KSR-2 (spanning amino acids 543-829 of SEQ ID NO:2) was generated with the FLAG epitope at its 3′ end (termed KSR-2-CD-FLAG) using PCR primers spanning the lysine residue at position 527 (5′-CACCATGGAGCAGCTGGAGATCGGCGAGCTCATT) (SEQ ID NO:248) and the FLAG tag sequence incorporated at the carboxyl terminus (3 ′-CTTCAGACGTCTCATGATGTTCCTACTGCTGCTATTCATCCGCGGCG) (SEQ ID NO:249) and the full-length KSR-2 as template DNA, and cloned into pcDNA-DEST-40 for mammalian expression.

293T cells (maintained in DMEM supplemented with 10% fetal bovine serum, 2% L-glutamine, and 1% penicillin/streptomycin) in logarithmic phase were transfected with 10 μg KSR-2-FLAG or KSR-2-CD-FLAG by calcium phosphate precipitation (ProFection™ Mammalian Transfection Systems, Promega, Madison, Wis.) as per the manufacturer's instructions. Untransfected cells served as a control. Cells were lysed in buffer containing protease and phosphatase inhibitors (0.2% NP-40, 25 mM Tris-HCl, pH 7.5, 150 mM NaCl, 1 mM DTT, 1 mM EDTA, 1 mM EGTA, 20 mM NaF, 1 mM Na₃VO₄, 1 mM glycerophosphate, and protease inhibitors) 48 h posttransfection. Cell lysates containing 50 μg protein were diluted in Laemmli buffer containing 2% b-mercaptoethanol and separated by SDS-PAGE on 4-7% polyacrylamide gels. The separated proteins were transferred to nitrocellulose membranes (Invitrogen) using a wet transfer electroblotter (Invitrogen). Following blocking overnight in 5% dry milk at room temperature, the membranes were probed with an anti-FLAG antibody (Eastman Kodak, New Haven, Conn.) at recommended dilution. Immunoreactive bands were detected using appropriate secondary antibodies conjugated to horseradish peroxidase (Amersham Biosciences, Piscataway, N.J.) at recommended dilution. Membranes were visualized using ECL Western Blotting Detection Reagents (Amersham). A band of ˜105 kDa was detected in lysates of cells transfected with KSR-2-FLAG, while a band of ˜37 kDa was detected in cells transfected with KSR-2-CD-FLAG. No bands were detected in untransfected cells.

### Example 3.2

### Identification of Proteins that Interact with KSR-2

To identify proteins that interact with human KSR-2, 293T cells were transfected with KSR-2-FLAG as described above. Cells transfected with FLAG vector alone served as a control. Cells were lysed as described above 48 h posttransfection. Protein concentrations were determined using the BCA protein assay. Cell lysates containing approximately 1 mg protein were subjected to anti-FLAG antibody immunoprecipitation followed by electrophoresis on a 2D gel resolved in only one dimension. The proteins were then transferred to nitrocellulose membranes and probed with various commercially available antibodies (Cell Signaling Technology, Beverly, Mass.) using the MN20SL Miniblotter (Immunetics, Cambridge, Mass.). Human KSR-2 was found to interact with P.ERK, MEK, P.MEK1, and P.Raf, proteins previously reported to interact with KSR (see Morrison, supra). Interestingly, KSR-2 also interacted with JNK, P.JNK, Cot/Tp12, and IRAK1, interactions that have not previously been reported for KSR. Probing of mock-transfected cells revealed no corresponding bands, indicating that the interactions between human KSR-2 and the above-mentioned proteins are specific. Human KSR-2 did not interact with ERK, P.P38, P.SEK1, or Myd 88.

### Example 3.3

### Human KSR-2 Specifically Interacts with Cot/Tp12 Through its Putative Kinase Domain

Because Cot/Tp12 is an oncogenic MAP kinase kinase kinase (MAP3K) that activates the MAPK pathway (Tsatsanis and Spandidos, Int. J. Mol. Med., 5:583 (2000)), the interaction of human KSR-2 with Cot/Tp12 was further assessed by cotransfecting 293T cells with a full-length Cot/Tp12 expression vector and KSR-2-FLAG or KSR-2-CD-FLAG as described above. Untransfected cells and cells transfected with single vectors served as controls. Cell lysates were subjected to either anti-Cot (Santa Cruz Biotechnology, Santa Cruz Calif.) or anti-FLAG antibody immunoprecipitation followed by SDS-PAGE. Proteins were transferred to nitrocellulose membranes and detected by reciprocal immunodetection (i.e., anti-Cot/Tp12 immunoprecipitation membranes were probed with anti-FLAG antibody, and anti-FLAG immunoprecipitation membranes were probed with anti-Tp12 antibody). Probing of anti-Cot/Tp12-immunoprecipitated lysates with anti-FLAG revealed either a ˜105 kD band corresponding to full-length human KSR-2 or a ˜37 kD band corresponding to the putative kinase domain of human KSR-2, indicating that KSR-2 interacts with Cot/Tp12 through its carboxy terminus. Similar results were obtained when anti-FLAG-immunoprecipitated lysates were probed with anti-Cot/Tp12 (a ˜50 kD band corresponding to Cot/Tp12 was identified). Truncation of the C-terminal 71 amino acids of Cot/Tp12 had no effect on its interaction with KSR-2. Probing of control lysates revealed no corresponding bands, indicating that the interaction between human KSR-2 and Cot/Tp12 is specific.

### Example 3.4

### Human KSR-2 Inhibits the Kinase Activity of Cot/Tp12

To determine the effect of KSR-2 on the kinase activity of Cot/Tp12, an in vitro kinase assay was performed using anti-FLAG-immunoprecipitated KSR-2 and purified recombinant Cot/Tp12. Following transfection of 293T cells with human KSR-2-FLAG, cell lysates were prepared as described above and subjected to immunoprecipitation using anti-FLAG and 20 μl 50% GammaBind Plus Sepharose beads (Amersham) that had been prewashed in lysis buffer. Bead bound-KSR-2 was eluted using competing FLAG peptide. The eluted KSR-2 was incubated with recombinant Cot/Tp12 or Raf enzyme (Upstate Biotechnology, Inc., Lake Placid, N.Y.) and 100 nM inactive GST-MEK1-His6 as substrate in kinase buffer (20 mM MOPS (pH. 7.2), 100 μM ATP, 5 mM EGTA, 20 mM MgCl₂, and protease and phosphatase inhibitors) in the presence of 5 μCi [γ-³³P] ATP. Various control kinase reactions were performed using the same buffer mix. Following 30 min at 30° C., the kinase reactions were stopped by the addition of LDS loading buffer (Invitrogen). The samples were boiled, centrifuged, and separated on a denaturing SDS-PAGE gel, and the radiolabeled proteins were visualized using the Molecular Imager FX (Bio-Rad, Hercules, Calif.). KSR-2 reduced Cot/Tp12 kinase activity in a dose-dependent manner, whereas Raf kinase activity was upregulated, indicating that KSR-2 selectively blocks Cot/Tp12-induced MEK phosphorylation, but not Raf-mediated MEK phosphorylation, in this system.

### Example 3.5

### Human KSR-2 Attenuates Cot/Tp12-Induced ERK Activation

To assess the downstream biological effects of the interaction between human KSR-2 and Cot/Tp12, 293T cells were cotransfected with Cot/Tp12 and KSR-2-FLAG or KSR-2-CD-FLAG expression vectors as described above. Untransfected cells served as a control. Following SDS-PAGE and blotting as described above, the membranes were probed with a polyclonal anti-P.ERK antibody (Cell Signaling Technology) at recommended dilution. No P.ERK was detected in cells transfected with hKSR-2-FLAG or hKSR-2-CD-FLAG alone, indicating that neither KSR-2 nor its putative kinase domain have any appreciable ERK activation potential. Cells transfected with Cot/Tp12 contained a very intense 42/44 kDa P.ERK doublet compared to untransfected cells, indicating that Cot/Tp12 activates ERK to P.ERK. Cotransfection of these cells with KSR-2-FLAG or KSR-2-CD-FLAG, however, reduced the intensity of the P.ERK doublet by ˜83%, indicating that KSR-2 blocks Cot/Tp12-mediated ERK activation. Coexpression of KSR-2 did not, however, affect Raf-mediated ERK activation, indicating that KSR-2 specifically inhibits only Cot/Tp12-mediated ERK activation. To rule out the possibility that the inhibitory effect on Cot/Tp12 by KSR-2 was due to its overexpression, hKSR-2 concentrations were titrated down while Cot/Tp12 levels remained constant. A dramatic decrease in the levels of Cot-induced ERK activation by KSR-2 was demonstrated in a dose-dependent manner. Consequently, KSR-2 inhibition of Cot/Tp12 is not an artifact of transient transfections.

### Example 3.6

### Human KSR-2 Blocks Cot/Tp12-Induced NF-κB Activation

Cot/Tp12 is reported to regulate NF-κB activation, either by targeting the IκB kinase (IKK) complex through the intermediate kinase NF-κB-inducing kinase (NIK) (Lin et al., Immunity, 10:271 (1999)) or by direct targeting and proteolysis of NF-κB inhibitory protein, p105 (Salmeron et al., J. Biol. Chem., 276:22215 (2001); Belich et al., Nature, 397:363 (1999)). To determine what role KSR-2 plays in this pathway, 293T cells containing a NF-κB/luciferase reporter plasmid (Promega, Madison, Wis.) were cotransfected with Cot/Tp12 and hKSR-2-FLAG or hKSR-2-CD-FLAG expression vectors (2.5-5 μg DNA/well of a 24-well plate, or 5-7 μg of DNA/well of 6-well plate) as described above. Cells transfected with single vectors served as controls. Cells were lysed 48 h posttransfection in 1× passive lysis buffer from the Dual-Luciferase Reporter Assay System (Promega). Twenty μl of cell lysate was transferred to 96-well plates (Lab Systems, Franklin, Mass.) specifically designed for luciferase assays. The Fluoroscan Ascent FL luminometer (Lab Systems) was programmed to perform a 2 sec premeasurement followed by a 10 sec reading of luciferase activity. Results were normalized using the renilla luciferase reporter plasmid pRL-TK supplied with the assay system. Cot/Tp12 alone strongly induced NF-κB-dependent luciferase activity (˜5- to 6-fold), whereas neither KSR-2 nor its putative kinase domain showed any NF-κB activation potential. Coexpression of Cot/Tp12 with KSR-2 or its putative kinase domain, however, blocked Cot/Tp12-induced NF-κB activation by ˜90%, indicating that KSR-2 is able to block Cot/Tp12-mediated cellular functions. Additionally, KSR-2 was able to reduce Cot/Tp12-mediated NF-κB levels in a dose-dependent manner, suggesting that KSR-2 inhibition of Cot/Tp12 is not an artifact of transient transfections.

### Example 3.7

### Human KSR-2 Blocks Cot/Tp12-Induced NF-κB Activation Independent of IKKβ

The specificity of the ability of KSR-2 to block Tp12-induced NF-κB activation was assessed in 293T cells containing a NF-κB/luciferase reporter plasmid cotransfected with IKKβ and hKSR-2-FLAG or hKSR-2-CD-FLAG expression vectors as described above. Measurement of luciferase activity (described above) revealed that IKKβ alone strongly induced NF-κB-dependent activation, whereas neither coexpression of IKKβ with KSR-2 nor with its putative catalytic domain had any effect on IKKβ-induced NF-κB activation. These results demonstrate the specificity of the observed Cot/Tp12 inhibition by KSR-2 in Example 3.6, and suggest that this inhibition is independent of the IKKβ signaling pathway.

### Example 3.8

### Human KSR-2 Attenuates Cot/Tp12-Induced IL-8 Production

Consistent with its ability to activate NF-κB, Cot/Tp12 has been shown in Tp12 knockout mice to regulate the production of various proinflammatory cytokines in monocytes and macrophages, including TNF-α, IL-1, and IL-6 (Elipoulos et al., EMBO J., 21:4831 (2002); Dumitru et al., Cell, 103:1071 (2000)). To assess the effect of KSR-2 on the ability of Cot/Tp12 to induce production of proinflammatory cytokines, HeLa cells (maintained as described above for 293T cells) were transfected with Cot/Tp12 and hKSR-2-FLAG or hKSR-2-CD-FLAG expression vectors using Fugene 6 (Roche, Indianapolis, Ind.) as per manufacturer's recommendations. Cells were either left untreated or treated with 100 ng/ml human recombinant TNF-α (R&D Systems, Minneapolis, Minn.) 48 h posttransfection. During treatments, cells were changed from DMEM containing 10% fetal bovine serum to DMEM containing 0.5% fetal bovine serum. The culture medium was collected 48 h posttransfection or posttreatment and frozen at −80° C. until further use. Cytokine levels were measured by using various ELISA kits from Biosource International (Carlsbad, Calif.) as per manufacturer's instructions. Depending on the initial observation, TNF-α-treated samples were diluted 5-fold in diluent buffer provided in the kit. Assays were run in duplicates, and samples were read at 450 nm in an ELISA plate reader (Wallac 1420 Multilable Counter, Perkin Elmer Life Sciences, Turku, Finland). Cytokine concentrations were determined by comparison to standards with a two-parameter curve fit analysis, with the dilution factor taken into account.

HeLa cells transfected with Cot/Tp12 exhibited a significant increase in IL-8 production, compared to untransfected cells. This is consistent with previous reports that the MAPK and NF-κB pathways are involved in IL-8 production (Neff et al., Cell. Microbiol., 3:703 (2001)). Transfection of Cot/Tp12 did not cause any increase in TNF-α, IL-1, or IL-6 production, suggesting that HeLa cells and monocytes and macrophages use different pathways for cytokine production. The Cot/Tp12-induced production of IL-8 was markedly reduced by coexpression of KSR-2. Although TNF-α treatment increased IL-8 production to an even greater extent in Cot/Tp12-transfected cells than in untreated transfected cells, coexpression of KSR-2 had no effect on the TNF-α-induced IL-8 production, suggesting that the Cot/Tp12 pathway may be independent of the TNF-α pathway.

To determine the specificity of IL-8 inhibition by KSR-2, the effect of KSR-2 on TAK1/TAB1-induced IL-8 production was assessed. In addition to Cot/Tp12, another major mechanism contributing to IL-8 gene expression is linked to TAK1, another MAP3K, and its coactivator TAB1 (Holtmann et al. (2001) J. Biol. Chem. 276:3508-16). Cotransfection of TAK1 and TAB1 expression vectors into HeLa cells resulted in a significant increase in IL-8 production. Coexpression of KSR-2 with TAK1/TAB1, however, had no effect on the increased IL-8 production, demonstrating that KSR-2 specifically inhibits only Cot/Tp12-induced IL-8 production.

Taken together, the results provide evidence for a novel regulatory role for KSR-2 in the inhibition of Cot/Tp12-mediated cellular functions, such as ERK and NF-κB activation, as well as IL-8 production. The results also suggest that KSR, a scaffold protein, may have dual regulatory roles, functioning as a positive or negative regulatory protein depending upon its protein targets.

What is claimed is:

1. An isolated nucleic acid molecule having the nucleotide sequence of SEQ ID NO:1.

2. An isolated nucleic acid molecule having the nucleotide sequence of SEQ ID NO:4.

3. The isolated nucleic acid molecule of claim 1 wherein said nucleic acid molecule is operably linked to at least one expression control sequence.

4. A host cell transformed or transfected with the nucleic acid molecule of claim 3.

5. The isolated nucleic acid molecule of claim 2 wherein said nucleic acid molecule is operably linked to at least one expression control sequence.

6. A host cell transformed or transfected with the nucleic acid molecule of claim 5.

7. An isolated nucleic acid molecule that specifically hybridizes under highly stringent conditions to the nucleotide sequence set forth in SEQ ID NO:1 or to the complement of the sequence set forth in SEQ ID NO:1.

8. An isolated nucleic acid molecule that specifically hybridizes under highly stringent conditions to the nucleotide sequence set forth in SEQ ID NO:4 or to the complement of the sequence set forth in SEQ ID NO:4.

9. An isolated nucleic acid molecule that encodes a protein having the amino acid sequence of SEQ ID NO:2.

10. An isolated nucleic acid molecule that encodes a protein having the amino acid sequence of SEQ ID NO:5.

11. An antisense oligonucleotide complementary to a mRNA corresponding to a nucleic acid molecule having the nucleotide sequence of SEQ ID NO:1, wherein said oligonucleotide inhibits production of KSR-2.

12. An antisense oligonucleotide complementary to a mRNA corresponding to a nucleic acid molecule having the nucleotide sequence of SEQ ID NO:4, wherein said oligonucleotide inhibits production of KSR-2.

13. An isolated gene having the nucleotide sequence of SEQ ID NO:3.

14. An isolated gene having the nucleotide sequence of SEQ ID NO:6.

15. An isolated allele of a nucleic acid molecule having the nucleotide sequence of SEQ ID NO:1.

16. An isolated allele of a nucleic acid molecule having the nucleotide sequence of SEQ ID NO:3.

17. An isolated allele of a nucleic acid molecule having the nucleotide sequence of SEQ ID NO:4.

18. An isolated allele of a nucleic acid molecule having the nucleotide sequence of SEQ ID NO:6.

19. An isolated protein having the amino acid sequence of SEQ ID NO:2, or an active fragment thereof.

20. An isolated protein having the amino acid sequence of SEQ ID NO:5, or an active fragment thereof.

21. An isolated antibody capable of binding to the protein of claim 19.

22. An isolated antibody capable of binding to the protein of claim 20.

23. A nonhuman transgenic animal in which all of the somatic and germ cells contain DNA comprising a nucleic acid molecule having the nucleotide sequence of SEQ ID NO:1.

24. A nonhuman transgenic animal in which all of the somatic and germ cells contain DNA comprising a nucleic acid molecule having the nucleotide sequence of SEQ ID NO:3.

25. A method of inhibiting Cot/Tp12-induced ERK activation in a cell population comprising transforming or transfecting said cell population with the nucleic acid molecule of claim 3.

26. A method of inhibiting Cot/Tp12-induced ERK activation in a cell population comprising transforming or transfecting said cell population with the nucleic acid molecule of claim 5.

27. A method of inhibiting Cot/Tp12-induced NF-κB activation in a cell population comprising transforming or transfecting said cell population with the nucleic acid molecule of claim 3.

28. A method of inhibiting Cot/Tp12-induced NF-κB activation in a cell population comprising transforming or transfecting said cell population with the nucleic acid molecule of claim 5.

29. A method of inhibiting NF-κB-mediated gene expression in a cell population comprising transforming or transfecting said cell population with the nucleic acid molecule of claim 3.

30. A method of inhibiting NF-κB-mediated gene expression in a cell population comprising transforming or transfecting said cell population with the nucleic acid molecule of claim 5.

31. A method of inhibiting IL-8 production in a cell population comprising transforming or transfecting said cell population with the nucleic acid molecule of claim 3.

32. A method of inhibiting IL-8 production in a cell population comprising transforming or transfecting said cell population with the nucleic acid molecule of claim 5.

33. A method of inhibiting expression of KSR-2 in a cell population comprising treating said cell population with the antisense oligonucleotide of claim 11.

34. A method of inhibiting expression of KSR-2 in a cell population comprising treating said cell population with the antisense oligonucleotide of claim 12.

35. A method of identifying a compound capable of inhibiting the activity of a KSR-2 protein comprising the steps of:
(a) contacting a first sample containing the KSR-2 protein with one of a plurality of test compounds; and
(b) comparing the activity of the KSR-2 protein in the first contacted sample with that of the KSR-2 protein in a second sample not contacted with the test compound,
wherein a decrease in the activity of the KSR-2 protein in the first sample, as compared with that in the second sample, identifies the compound as an inhibitor of KSR-2 protein activity.

36. The method of claim 35, wherein the KSR-2 protein is the protein of claim 19.

37. The method of claim 35, wherein the KSR-2 protein is the protein of claim 20.

38. A method of identifying a compound capable of increasing the activity of a KSR-2 protein comprising the steps of:
(a) contacting a first sample containing the KSR-2 protein with one of a plurality of test compounds; and
(b) comparing the activity of the KSR-2 protein in the first contacted sample with that of the KSR-2 protein in a second sample not contacted with the test compound,
wherein an increase in the activity of the KSR-2 protein in the first sample, as compared with that in the second sample, identifies the compound as an activator of KSR-2 protein activity.

39. The method of claim 38, wherein the KSR-2 protein is the protein of claim 19.

40. The method of claim 38, wherein the KSR-2 protein is the protein of claim 20.

41. A method of inhibiting expression of KSR-2 in a cell population comprising treating said cell population with a siRNA molecule targeted to a mRNA corresponding to the isolated nucleic acid molecule of claim 1.

42. The method of claim 41, wherein the siRNA molecule is selected from the group consisting of siRNA molecules shown in FIG. 1.

43. A method of inhibiting expression of KSR-2 in a cell population comprising treating said cell population with a siRNA molecule targeted to a mRNA corresponding to the isolated nucleic acid molecule of claim 2.

44. A siRNA molecule that inhibits the expression of KSR-2.

45. A siRNA molecule of claim 44, wherein the siRNA molecule is selected from the group consisting of siRNA molecules shown in FIG. 1.
